(19) United States
(12) Patent Application Publication
(10) Pub. No.: US 2004/0186585 A1
(43) Pub. Date: Sep. 23, 2004

(54) Sphere-on-sphere ankle prosthesis

(76) Inventor: Lawrence Feiwell, Huntington Beach, CA (US)

Correspondence Address: Attention: Kenneth S. Barrow, Centerpulse USA Inc., Suite 1000, 12 Greenway Plaza, Houston, TX, 77046, US

(21) Appl. No.: 10/394,672

(22) Filed: Mar. 21, 2003

(51) Int. Cl. A61F 2/42

(52) U.S. Cl. 623/21.18

(57) ABSTRACT

An ankle prosthesis using sphere-on-sphere articulating components. The prosthesis comprises two separate components, a tibial component and a talar component. Each component has an articulating surface and a fixation surface. The articulating surfaces form a spherical interface with one component having a spherical concave surface and the other component having a spherical convex surface.

### FIELD OF THE INVENTION

The disclosure herein generally relates to implantable orthopedic prostheses and, more particularly, to fixed bearing ankle prostheses using sphere-on-sphere articulating components.

### BACKGROUND OF THE INVENTION

In the United States alone, thousands of ankle arthrodesis and ankle arthroplasty procedures are performed each year. Osteoarthritis arthritis, rheumatoid arthritis, and post-traumatic arthritis are the most common reasons for these procedures. In some severe arthritic cases, cartilage and synovium surrounding the ankle wear down so underlying bones grind directly on each other. Arthritis of the ankle, in all these forms, can cause severe pain and disability for the patient.

Patients have several surgical treatment options for arthritis of the ankle. These options include ankle arthroscopy, ankle arthrotomy and debridement (opening the joint and removing bone spurs), osteotomy (cutting bone to realign the joint), ankle fusion or arthrodesis (removing the joint and making it stiff), and total ankle arthroplasty (replacing the joint with an ankle prosthesis).

To date, ankle arthrodesis is one widely accepted procedure for treating arthritis in the ankle. This procedure though, has many shortcomings, such as infection, skin slough, nerve injury or entrapment, and nonunion or mal-union. Further yet, a big disadvantage with this procedure is that the patient must sacrifice ankle motion and mobility to relieve pain. This sacrifice, in turn, can increase stress on other joints. Some surgeons, for instance, believe that arthrodesis compromises the knee and hip joints because the body attempts to compensate for the lost range of motion at the ankle. Given these shortcomings, more focus has been devoted to ankle arthroplasty as an alternative to ankle arthrodesis.

In ankle arthroplasty, portions of the natural ankle joint are replaced with prosthetic components. These components include a tibial component implanted into the end of the tibia and a talar component implanted into the end of the talus. These two components include articulation surfaces that articulate against each other in an attempt to simulate the natural articulating motion between the tibia and talus. The components are made of materials that exhibit a low coefficient of friction when they articulate during normal range of motion.

Much effort has been devoted to designing ankle prostheses that restore normal, pain-free functions to the ankle joint. To date, these prostheses have had limited success. The first generation of ankle prosthesis was not designed to emulate the biomechanical characteristics of the natural joint. The designs required excessive bone resection and ultimately had a high rate of long term failure.

A second generation of ankle prosthesis improved on the first generation but still exhibited numerous disadvantages. Many of the designs have lead to complications and failures. In short, these prostheses have failed to emulate the natural kinematics of the ankle joint.

It therefore would be advantageous to provide a new generation of ankle prostheses that have significant advantages over the first and second generation of prostheses.

### SUMMARY OF THE INVENTION

The present invention is directed toward ankle prostheses using sphere-on-sphere articulating components. In the preferred embodiment, these prostheses are fixed bearing and cement retained. The prostheses generally comprise two separate components, a tibial component and a talar component. Each component has an articulating surface and a fixation surface oppositely disposed from the articulating surface. The fixation surface is adapted to engage and fix to bone. The articulating surfaces form a spherical interface. One component has a spherical concave surface, and the other component has a spherical convex surface. These surfaces are sized and shaped to mate and slideably articulate with each other.

One important advantage of the present invention is that the joint motion surface of the ankle prosthesis uses sphere-on-sphere articulating surfaces. This spherical interface between articulating surfaces is a critical component of the present invention. This interface more closely emulates the natural kinematics of the ankle joint and results in numerous advantages over prior designs.

As one advantage, the design decreases torsional forces across the ankle joint. These forces can result from a slight mal-alignment in any plane or high physiological stresses across the ankle joint.

As another advantage, the spherical design increases or maximizes the surface area of contact between the tibial and talar prosthetic component. As such, weight bearing loads are more evenly distributed across a larger area. This distribution is extremely important at the ankle joint. Here, loads are often eccentric (i.e., not central and evenly distributed). An uneven distribution of force causes compression on one side of the bone and a lift-off force on the opposite side. Expanding the surface area of articulating surfaces decreases the overall force per unit area. Further yet, the increased spherical area decreases angular stress or shearing that can cause micromotion. The spherical articulating surfaces are thus more resistant to the negative affects of eccentric forces, shear stresses, and micromotion.

As yet another advantage, one embodiment uses metal-on-metal articulating surfaces. These components can be manufactured to have a thin or minimal thickness yet exhibit high strength. The thin components result in nominal resection of bone, leaving more natural bone on the talus and tibia. As a result, the fixation between the bone engaging surfaces of the implant and cortical bone is strong. The added strength in this fixation prevents or reduces subsidence and ultimately early failure of the prosthesis. In an extreme case where failure still occurs at a later date, sufficient bone will remain for other operative procedures, such as a revision surgery or fusion. This additional strength also makes the prosthesis less prone to fail mechanically. A strong prosthesis is particularly important at this joint since the forefoot acts as lever to magnify compressive forces between the tibial and talus. These forces, for example, can be 5-7 times the weight of the patient.

As yet another advantage, the spherical design provides strong bone support. Good support at the ankle joint is fundamental for success of the prosthesis. Poor support often leads to subsidence, loosening, and ultimately failure. This support is notably critical at the talus since it is anatomically small and provides a limited area for fixation.

As still yet another advantage, another embodiment of the present invention uses metal-on-polymeric articulating surfaces. Here, the talar component is made of polyethylene and the tibial component is made of metal. The metallic talar component has the same advantages as the talar component in the metal-on-metal embodiment, such as high strength and reduced bone resection. The polymeric component can be formed of a highly cross-linked UHMWPe that exhibits high strength and extremely low friction and wear properties.

Other advantages and benefits of the present invention will be apparent from the accompanying drawings and detailed disclosure of the invention.

### BRIEF DESCRIPTION OF THE DRAWINGS

FIG. 1A is a top perspective view of a convex component of the ankle prosthesis.

FIG. 1B is a bottom perspective view of the convex component of FIG. 1A.

FIG. 2A is a top perspective view of a concave component of the ankle prosthesis.

FIG. 2B is a bottom perspective view of the concave component of FIG. 2A.

FIG. 3 is a side view of the concave component attached to a tibia and the convex component attached to a talus.

FIG. 4 is a side view of the convex component attached to the tibia and the concave component attached to the talus.

FIG. 5 is an ankle prosthetic system having multiple sized concave and convex components that are connectable together.

### DETAILED DESCRIPTION

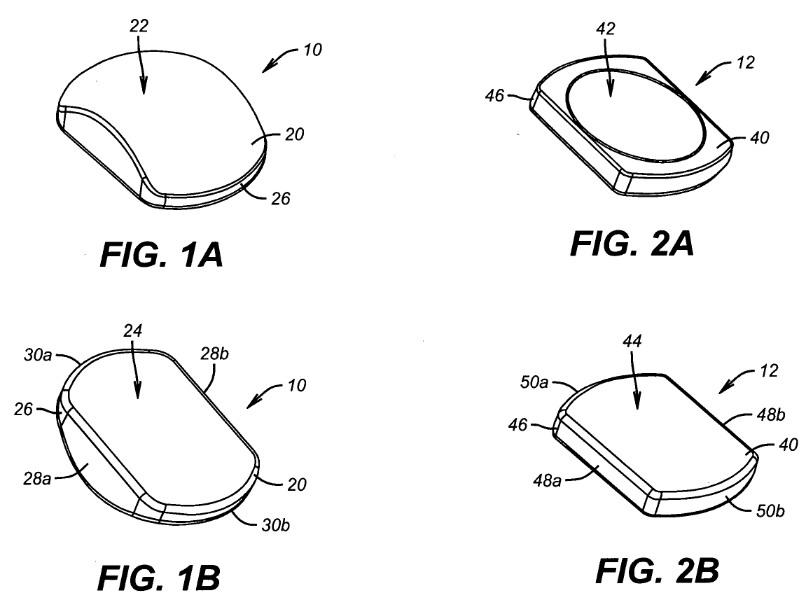

FIGS. 1A, 1B, 2A, and 2B together show the ankle prosthesis of the present invention. The prosthesis comprises two separate and distinct components, a convex component 10 and a concave component 12.

The convex component 10 has a body 20 with an articulating surface 22 on one side and a fixation surface 24 oppositely disposed on the other side. An outer wall 26 extends around a perimeter and includes two straight walls 28a and 28b and two rounded walls 30a and 30b. The perimeter forms a square or rectangular shape with rounded end walls. Articulating surface 22 has a smooth outer surface that has a spherical, convex shape. Preferably, surface 22 is symmetric with a continuous contour. Fixation surface 24 is flat or planar and may be adapted to be cement retained, screw retained, or the like.

The concave component 12 has a body 40 with an articulating surface 42 on one side and a fixation surface 44 oppositely disposed on the other side. An outer wall 46 extends around a perimeter and includes two straight walls 48a and 48b and two rounded walls 50a and 50b. The perimeter forms a square or rectangular shape with rounded end walls. Articulating surface 42 has a smooth outer surface that has a spherical, concave shape. Preferably, surface 42 is symmetric with a continuous contour. Fixation surface 44 is flat or planar and may be adapted to be cement retained, screw retained, or the like.

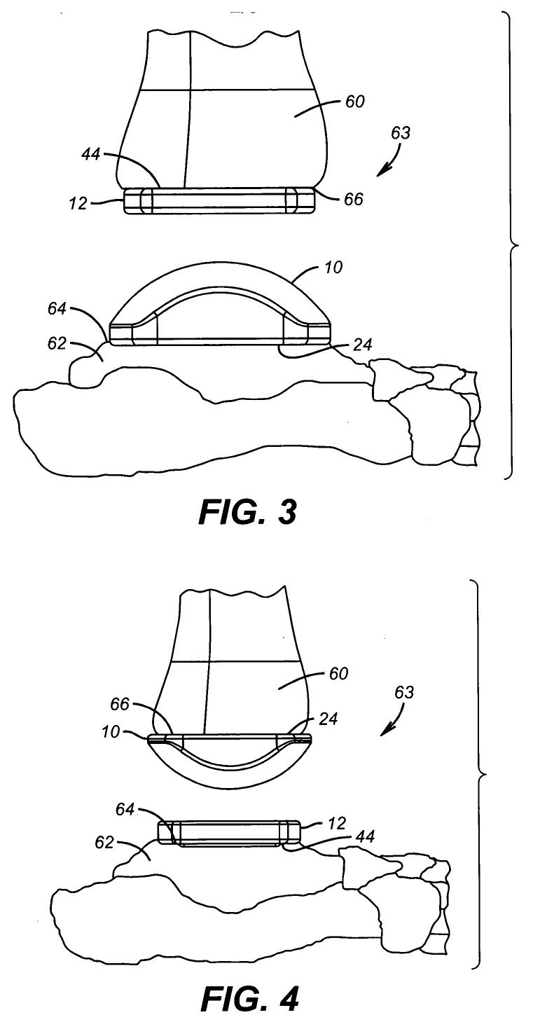

Looking to FIG. 3, concave component 12 is connected to the distal end of the tibia 60, and convex component 10 is connected to the talus 62. Together, these two components form the ankle prosthesis at the ankle joint 63 of a patient. More specifically, the fixation surface 24 of convex component 10 is engaged and connected to a planar surface 64 of talus 62. Likewise, fixation surface 44 of concave component 12 is engaged and connected to a planar surface 66 of tibia 60.

The articulating surfaces 22 and 42 are sized and shape to engage and slideably articulate with each other. During relative motion between the two surfaces, a portion of articulating surface 22 fits inside the concave cavity and smoothly rolls against articulating surface 42. During motion then, the two components articulate with each other with a sphere-on-sphere articulation. As noted, this spherical interface between the articulating surfaces is a critical component of the present invention.

As best shown in FIGS. 1A, 1B, 2A, 2B, and 3, the concave and convex components have substantially the same length and width. The components are thus shaped to articulate against each other in the naturally confined area of the ankle joint of a patient.

One advantage of the present invention is that the convex and concave components can be used on either the tibia or talus. In contrast to FIG. 3, the embodiment in FIG. 4 shows concave component 12 connected to the talus 62 and convex component 10 connected to the tibia 60. Together, these two components form the ankle prosthesis at the ankle joint 63 of a patient. More specifically, the fixation surface 24 of convex component 10 is engaged and connected to the planar surface 66 of tibia 60. Likewise, fixation surface 44 of concave component 12 is engaged and connected to the planar surface 64 of talus 62.

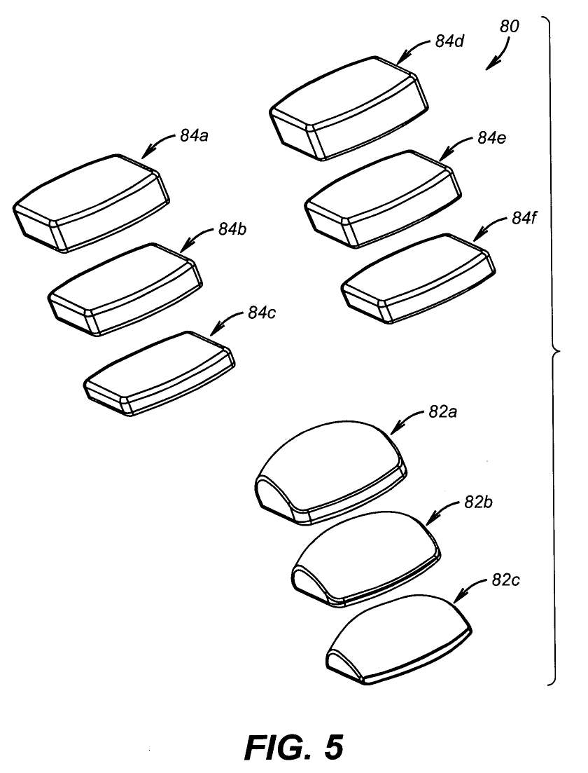

The ankle prostheses of the present invention can be utilized in a system comprising a plurality of differently sized concave and convex components that are interchangeable and useable to form various ankle prostheses. One skilled in the art will appreciate that this system includes the various embodiments taught in this disclosure. One such system 80 is shown in FIG. 5.

System 80 includes three differently sized convex components 82a-82c and six differently sized concave components 84a-84f. The components can be generally sized to have small, medium, and large sizes. Each size can, for example, vary in thickness. More specifically, convex components 82a-82c can have sizes of neutral, +2mm, and +4mm. Further, concave components 84a-84f can have sizes of neutral, +2mm, +4mm wherein each thickness further comes either in a shallow articulation surface (−60° sphere) or in a deep articulation surface (−75° sphere).

As noted, one of the advantages of the present invention is that the components can be fabricated from metal and/or polymer. These metals include Cobalt Chrome (CoCr), Titanium alloy, Commercially pure Titanium, Stainless Steel, Titanium nitride on CoCr, or Diamond-like Coating on Titanium or CoCr. Further, these polymers include many commercially available forms known in the art, such as polyethylene and UHMWPe. Examples of suitable materials are Metasul® and Durasul® articulation components manufactured by Centerpulse Orthopedics Inc. of Austin, Tex.

The figures of the present invention teach ankle prostheses with talar and tibial fixation surfaces that are adapted to engage or connect to bone with bone cement. One skilled in the art will appreciate that these fixation surface could equally be adapted to engage and connect to bone without the use of bone cement and instead use bone integration and/or bone screws. Techniques for preparing surfaces for bone integration are known in the art and are within the scope of the invention.

Although illustrative embodiments have been shown and described, a wide range of modifications, changes, and substitutions is contemplated in the foregoing disclosure; and some features of the embodiments may be employed without a corresponding use of other features. Accordingly, it is appropriate that the appended claims be construed broadly and in a manner consistent with the scope of the embodiments disclosed herein.

What is claimed is:

1) An ankle prosthesis, comprising:
a talar component having a body with a talar fixation surface and an oppositely disposed talar articulating surface, wherein the talar fixation surface is adapted to engage a natural talus and the talar articulation surface has a spherical convex shape; and
a tibial component having a body with a tibial fixation surface and an oppositely disposed tibial articulating surface, wherein the tibial fixation surface is adapted to engage a natural tibia and the tibial articulation surface has a spherical concave shape that is adapted to articulate with the talar articulating surface.

2) The ankle prosthesis of claim 1 wherein the talar and tibial components are both made of metal.

3) The ankle prosthesis of claim 1 the talar component is made of a different material than the tibial component.

4) The ankle prosthesis of claim 3 wherein the talar component is made of polymer and the tibial component is made of metal.

5) The ankle prosthesis of claim 1 wherein talar and tibial articulating surfaces are sized and shape to slideably articulate with each other.

6) The ankle prosthesis of claim 1 wherein the talar and tibial components are fixed bearing.

7) An ankle prosthesis, comprising:
a talar component having a body with a talar fixation surface and an oppositely disposed talar articulating surface, wherein the talar fixation surface is adapted to connect to a natural talus and the talar articulation surface has a spherical concave shape; and
a tibial component having a body with a tibial fixation surface and an oppositely disposed tibial articulating surface, wherein the tibial fixation surface is adapted to connect to a natural tibia and the tibial articulation surface has a spherical convex shape that is adapted to articulate with the talar articulating surface.

8) The ankle prosthesis of claim 7 wherein both the talar and tibial components are made of metal.

9) The ankle prosthesis of claim 7 wherein the talar component is made of polymer and the tibial component is made of metal.

10) The ankle prosthesis of claim 7 wherein the talar component is made of a different material than the tibial component.

11) A prosthetic ankle system, comprising:
a plurality of differently sized talar components, wherein each talar component has a talar fixation surface adapted to engage a natural talus and a talar articulating surface; and
a plurality of differently sized tibial components, wherein each tibial component has a tibial fixation surface adapted to engage a natural tibia and a tibial articulating surface adapted to engage the talar articulating surface with sphere-on-sphere articulation, wherein at least one talar component and at least one tibial component together form an ankle prosthesis adapted for use at an ankle joint.

12) The prosthetic ankle system of claim 11 including at least three differently sized talar components and three differently sized tibial components, wherein each talar component can articulate with each tibial component and form an ankle prosthesis.

13) The prosthetic ankle system of claim 12 wherein each talar component has a different thickness and each tibial component has a different thickness.

14) The prosthetic ankle system of claim 13 wherein the tibial components have a concave tibial articulating surface and the talar components have a convex talar articulating surface.
